(19) United States
(12) Patent Application Publication
(10) Pub. No.: US 2025/0256963 A1
(43) Pub. Date: Aug. 14, 2025

(54) SYNTHESIS OF NANO-HYDROXYAPATITE FROM DECARBONIZED EGGSHELLS

(71) Applicant: KING FAHD UNIVERSITY OF PETROLEUM AND MINERALS, Dhahran (SA)

(72) Inventor: Morsi Mohamed Mahmoud Morsi AHMED, Dhahran (SA)

(73) Assignee: KING FAHD UNIVERSITY OF PETROLEUM AND MINERALS, Dhahran (SA)

(21) Appl. No.: 18/439,162

(22) Filed: Feb. 12, 2024

(51) Int. Cl. C01B 25/32; B82Y 5/00

(52) U.S. Cl. CPC C01B 25/327; B82Y 5/00; C01P 2004/61

(57) ABSTRACT

A method of synthesizing a hydroxyapatite (HAp) product from eggshells, including reacting eggshells with an inorganic base in a polar solvent to decarbonize the eggshells and form a calcium hydroxide material and a sodium carbonate material and mixing the calcium hydroxide material, in a polar solvent at a pH of greater than 10, with a phosphate salt to form a mixture including the hydroxyapatite product. A ratio of the calcium hydroxide material to the phosphate salt is from 1:1 to 5:1. The method further includes microwave irradiating the mixture for 1 to 10 minutes (min) at a temperature from 80 to 120 degrees Celsius (° C.), filtering the mixture to collect the hydroxyapatite product, and further drying the hydroxyapatite product.

### STATEMENT OF ACKNOWLEDGEMENT

Support provided by the Deanship of Research Oversight and Coordination (DROC) at King Fahd University of Petroleum and Minerals and the Interdisciplinary Research Center for Advanced Materials (IRC-AM), King Fahd University of Petroleum and Minerals, Saudi Arabia, through Project INAM2302 is gratefully acknowledged.

### BACKGROUND

### Technical Field

The present disclosure is directed towards a hydroxyapatite product, particularly a nano-hydroxyapatite (HAp) synthesized from decarbonized eggshells, and a method of preparation thereof.

### Description of Related Art

The “background” description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Various natural waste materials can and should be utilized effectively via sustainable waste management routes. While it may seem that some of these waste materials are useless at certain stages, their unexploited potential application is now being revealed. Most recently, the world has focused on waste management to convert generated waste into more valuable materials due to associated environmental benefits. Conversely, converting those wastes into high-value-added products will aid and enhance sustainable development. Eggshell waste is considered one of the most common food wastes. In 2021, it was reported that around 250,000 tons of eggshells were formed annually. This worldwide reported number is thought to increase, where the consumption trend of chicken eggs as a relatively affordable protein source will increase that waste amount. The challenges associated with eggshell waste generation will be directly related to and affect our environment and economy. Eggshell waste material sometimes ends up in landfills or in a low-value application. Eggshells are about 94 percent by weight (wt. %) calcite and calcium carbonate, which makes them potential materials for producing hydroxyapatite (HAp). Other components of eggshells (in wt. %) may be magnesium carbonate 1%, calcium phosphate 1%, and organic debris 4%. According to European Union regulations, eggshell waste is considered hazardous waste [Quina, M. J., M. A. R. Soares, and R. Quinta-Ferreira, Applications of industrial eggshell as a valuable anthropogenic resource. Resources, Conservation and Recycling, 2017. 123: p. 176-186]; therefore, converting eggshell waste into valuable materials, such as HAp, for different applications is of great interest. The utilization of eggshells to produce HAp bioceramic materials was investigated extensively over the years using different routes via either direct calcination or acidic or alkaline treatments of eggshells without decarbonization. Eggshell utilization is thought to reduce such waste and convert it into a useful product, like HAp, that may be used in various applications such as bone repair, tissue engineering, drug delivery, and the like.

HAp is a notable biomaterial due to its similar structure to human bones and teeth and its unique ability to be integrated with tissues. It is an attractive material for many biomedical applications, such as bone substitute materials in orthopedics and dentistry, due to its excellent biocompatibility, bioactivity, and osteoconduction properties. It is an inorganic component found naturally in human hard tissues and other sources, such as fish bones, coral, eggshells, chicken bones, and the like. Stoichiometric HAp has the molecular formula of Ca₅(PO₄)₃OH, which has a hexagonal dipyramidal crystal structure with a space group P6_{3/m}, as shown in FIG. 1A with the lattice parameters and unit cell volume shown in Table 1, [Ardanova, L. I., et al., Isomorphous Substitutions of Rare Earth Elements for Calcium in Synthetic Hydroxyapatites. Inorganic Chemistry, 2010. 49(22): p. 10687-10693] where its unit cell has two formula units, so it is usually written as Ca₁₀(PO₄)₆(OH)₂. As shown in the HAp unit cell, four Ca atoms are surrounded by nine O atoms from phosphate moieties, while the other six Ca atoms are surrounded by the other six O atoms from phosphate moieties. HAp can include other element traces, such as phosphite ions (PO₃³⁻), chloride ions (Cl⁻), fluoride ions (F), and hydroxyl ions (OH⁻), depending on their source.

TABLE 1
Crystal structure and lattice parameters of HAp.

| Crystal | Space | | | | | | | Unit cell |
|---|---|---|---|---|---|---|---|---|
| System | Group | a (Å) | b (Å) | c (Å) | α | β | γ | volume (Å³) |
| Hexagonal | P6_{3/m} | 9.41898 | 9.41898 | 6.88119 | 90° | 90° | 120° | 528.6910 |

Microwave (MW) radiation has been used as a promising processing tool for many materials and products manufacturing because of its several advantages over commonly used traditional processing techniques [Singh, C., V. Khanna, and S. Singh, Sustainability of microwave heating in materials processing technologies. Materials Today: Proceedings, 2023. 73: p. 241-248]. It is a powerful and different tool used to process several materials with different applications where an improvement in the material's performance and properties can be observed. MW processing can lead to shorter processing time, less energy consumption, selective heating, self-limiting reactions, and other benefits.

A circular economy is based on sustainability, recyclability, and benefiting from waste materials via converting them into high-value-added materials in a closed loop, where the “3R” principles (reduce, reuse, and recycle) are successfully implemented. Based on that, waste should be turned into new products to increase sustainable economic development. Hence, industrial-scale conversion of eggshell waste into HAp material is thought to yield a higher economic value than the expense of usual removal processes. At the same time, it will decrease the danger of pathogen spread and will also reduce the dumping costs. It will not only result in environmental profits, but also greatly help towards achieving the goal of a sustainable future and life.

Although different methods have been used in the past for the synthesis of HAp from eggshell waste, there still exists a need to develop new methods to produce high-value-added HAp material from eggshell waste in an environmentally and economically appealing manner. According, an objective of the present disclosure is to develop a hydroxyapatite product from decarbonized eggshells using microwave radiation to overcome the limitations of the art.

### SUMMARY

In an exemplary embodiment, a method of synthesizing a hydroxyapatite product is described. The method includes reacting eggshells with an inorganic base in a polar solvent to decarbonize the eggshells and form a calcium hydroxide material and a sodium carbonate material and mixing the calcium hydroxide material, in a polar solvent at a pH of greater than 10, with a phosphate salt to form a mixture including the hydroxyapatite product. A ratio of the calcium hydroxide material to the phosphate salt is from 1:1 to 5:1. The method further includes microwave irradiating the mixture for 1 to 10 minutes (min) at a temperature from 80 to 120 degrees Celsius (° C.), filtering the mixture to collect the hydroxyapatite product, and further drying the hydroxyapatite product.

In some embodiments, 94 to 98 percent by weight of the eggshells is converted into the calcium hydroxide material and the sodium carbonate material based on an initial weight of the eggshells.

In some embodiments, the hydroxyapatite product has a particle shape in the form of nanorods.

In some embodiments, the nanorods are agglomerated.

In some embodiments, the nanorods have an average size of 10 to 40 nanometers (nm).

In some embodiments, the nanorods have the longest dimension of 10 to 150 nm.

In some embodiments, the hydroxyapatite product includes carbon in an amount of 15 to 25 atomic percent (at. %), oxygen in an amount of 60 to 70 at. %, calcium in an amount of 5 to 15 at. %, and phosphorous in an amount of 3 to 10 at. % based on a total atom count of the hydroxyapatite product.

In some embodiments, the hydroxyapatite product has a crystalline apatite structure.

In some embodiments, the hydroxyapatite product has a calcium-to-phosphorous molar ratio of 1.5 to 1.9.

In some embodiments, the hydroxyapatite product has a first d-spacing value of 0.330 to 0.340 nm.

In some embodiments, the hydroxyapatite product has a second d-spacing value of 0.280 to 0.285 nm.

In some embodiments, the eggshells are in the form of a powder with an average crystallite size of 60 to 90 nm.

In some embodiments, the reaction occurs at ambient temperature and ambient pressure for 5 to 20 minutes.

In some embodiments, a ratio of the eggshells to the inorganic base to the polar solvent is from 5:30:50 to 10:40:60 based on a percent by weight of the total weight of the reacting.

In some embodiments, the calcium hydroxide material and the sodium carbonate material are separated by filtration.

In some embodiments, the method includes microwave irradiating the mixture at a frequency of 2 to 3 gigahertz (GHz).

In some embodiments, the method includes microwave irradiating the mixture at a power of 600 to 1000 watts (W).

In some embodiments, the phosphate salt is ammonium dihydrogen phosphate.

In some embodiments, the inorganic base is a hydroxide base.

In some embodiments, the method includes drying the hydroxyapatite product at 100 to 120° C. for 1 to 5 hours (h).

These and other aspects of the non-limiting embodiments of the present disclosure will become apparent to those skilled in the art upon review of the following specific non-limiting embodiments of the disclosure in conjunction with the accompanying drawings. The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1A depicts a stoichiometric hydroxyapatite (HAp) unit cell, according to certain embodiments;

FIG. 1B is a method flowchart for synthesizing a HAp product, according to certain embodiments;

FIG. 2 shows a schematic chart for an eggshell decarbonization process to obtain sodium carbonate (Na₂CO₃) and calcium hydroxide (Ca(OH)₂) precursors, according to certain embodiments;

FIG. 3 is a microwave synthesis setup of the HAp product from decarbonized eggshells, according to certain embodiments;

FIG. 4 depicts an X-ray diffraction (XRD) pattern of an eggshell powder, according to certain embodiments;

FIG. 5 depicts the XRD pattern of the microwave (MW) synthesized HAp product, using decarbonized eggshells, according to certain embodiments;

FIG. 6 is a Fourier-transform infrared (FTIR) spectrum of the MW synthesized HAp product using decarbonized eggshells, according to certain embodiments;

FIG. 7A is a scanning electron microscopy (SEM) image of the MW synthesized HAp product using decarbonized eggshells, at 1 μm magnification, according to certain embodiments;

FIG. 7B is an SEM image of the MW synthesized HAp product, using decarbonized eggshells, at 500 nm magnification, according to certain embodiments;

FIG. 7C is an SEM image of the MW synthesized HAp product using decarbonized eggshells, at 400 nm magnification, according to certain embodiments;

FIG. 7D shows an energy dispersive X-ray spectroscopy (EDS) of the MW synthesized HAp product, according to certain embodiments;

FIG. 8A is a transmission electron microscopy (TEM) image of the MW synthesized HAp product using decarbonized eggshells, at 500 nm magnification, according to certain embodiments;

FIG. 8B is a TEM image of the MW synthesized HAp product using decarbonized eggshells, at 200 nm magnification, according to certain embodiments;

FIG. 8C is a TEM image of the MW synthesized HAp product using decarbonized eggshells, at 100 nm magnification, according to certain embodiments;

FIG. 8D is a TEM image of the MW synthesized HAp product using decarbonized eggshells, at 50 nm magnification, according to certain embodiments;

FIG. 8E is a selected area electron diffraction (SAED) pattern of the MW synthesized HAp product using decarbonized eggshells, according to certain embodiments; and

FIG. 8F is a field emission transmission electron microscopy (FE-TEM) image showing interplanar lattice fringes of the MW synthesized HAp product using decarbonized eggshells, according to certain embodiments.

### DETAILED DESCRIPTION

In the following description, it is understood that other embodiments may be utilized, and structural and operational changes may be made without departure from the scope of the present embodiments disclosed herein.

When describing the present disclosure, the terms used are to be construed in accordance with the following definitions, unless a context dictates otherwise. Embodiments of the present disclosure will now be described more fully hereinafter with reference to the accompanying drawings wherever applicable, in that some, but not all embodiments of the disclosure are shown.

Reference will now be made to specific embodiments or features, examples of which are illustrated in the accompanying drawings. In the drawings, whenever possible, corresponding or similar reference numerals will be used to designate identical or corresponding parts throughout the several views. Moreover, references to various elements described herein are made collectively or individually when there may be more than one element of the same type; however, such references are merely exemplary in nature. It may be noted that any reference to elements in the singular may also be constructed to relate to the plural and vice-versa without limiting the scope of the disclosure to the exact number or type of such elements unless set forth explicitly in the appended claims. Further, as used herein, the use of the singular includes plural and the words “a,” “an,” and the like generally carry a meaning of “one or more,” and “at least one,” unless stated otherwise.

Furthermore, the terms “approximately,” “approximate,” “about,” and similar terms generally refer to ranges that include the identified value within a margin of 20%, 10%, or preferably 5%, and any values therebetween.

The use of the terms “include,” “includes,” including,” “have,” “has,” or “having” should be generally understood as open-ended and non-limiting unless specifically stated otherwise.

As used herein, “nanoparticles” refers to particles having a particle size of 1 nanometer (nm) to 500 nm within the scope of the present disclosure. The nanoparticles (NPs) may exist in various morphological shapes such as nanowire, nanocrystals, nanorectangles, nanotriangles, nanopentagons, nanohexagons, nanoprisms, nanorods, nanodisks, nanocubes, nanoribbons, nanoblocks, nanobeads, nanotoroids, nanodiscs, nanobarrels, nanogranules, nanowhiskers, nanoflakes, nanofoils, nanopowders, nanoboxes, nanostars, nanotetrapods, nanobelts, nano-urchins, nanoflowers, the like, and mixtures thereof.

As used herein, the term “particle size” may be thought of as the length or longest dimension of a particle.

As used herein, the term “sonication” refers to the process in which sound waves are used to agitate particles in a solution. Sonication may also refer to the process of applying sound energy to agitate particles and/or discontinuous fibers in a liquid.

As used herein, the term “microwave(s) (MW)” refers to a type of electromagnetic radiation with wavelengths ranging from about 30 centimeters (cm) to one millimeter (mm), and frequencies between 0.3 gigahertz (GHz) and 300 GHz, respectively. Microwaves are also referred to as microwave radiation and/or microwave irradiation. Microwave irradiation may be used to accelerate a variety of chemical reaction. The increase in frequency of molecular vibrations during microwave irradiation accelerates the chemical reactions.

As used herein, the term “hydroxyapatite (HAp)” refers to an inorganic mineral that has a typical apatite lattice structure of A₁₀(BO₄)₆C₂ where A, B, and C are defined be Ca, P, and OH, respectively. Pure HAp contains 39.68 percent by weight calcium and 18 percent by weight phosphorus resulting in a Ca/P mole ratio of 1.67. HAp is a mineral form of calcium apatite with the formula Ca₅(PO₄)₃(OH), often written Ca₁₀(PO₄)₆(OH)₂ to indicate that the crystal unit cell contains two entities. It is the hydroxyl endmember of the complex apatite group. It crystallizes in the hexagonal crystal system.

A weight percent of a component, unless specifically stated to the contrary, is based on the total weight of the formulation or composition in which the component is included. For example, if a particular element or component in a composition or article is said to have 5 percent by weight (weight percent) (wt. %), it is understood that this percentage is in relation to a total compositional percentage of 100%.

The present disclosure is intended to include all isotopes of a given compound or formula, unless otherwise noted. The present disclosure is intended to include all hydration states of a given compound or formula, unless otherwise noted or when heating a material.

Aspects of the present disclosure are directed toward the microwave (MW) synthesis of a nano-hydroxyapatite (HAp) product from decarbonized eggshells, which is a natural source of calcium carbonate (CaCO₃). This process aligns with the “3R” concept (reduce, reuse, and recycle) of circular economy and fulfills the demand for sustainability, leading to a better quality of life.

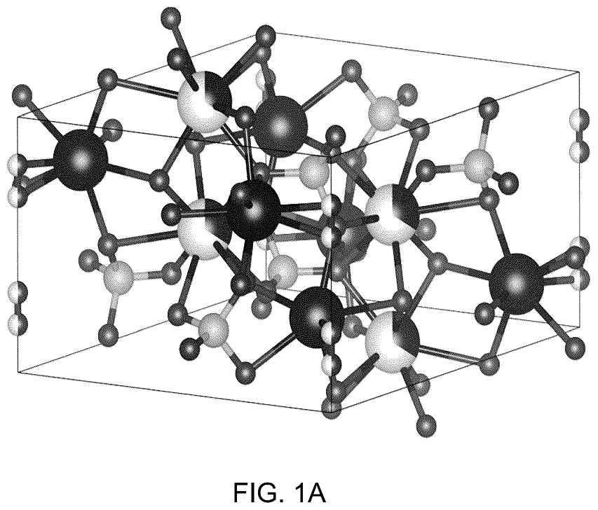

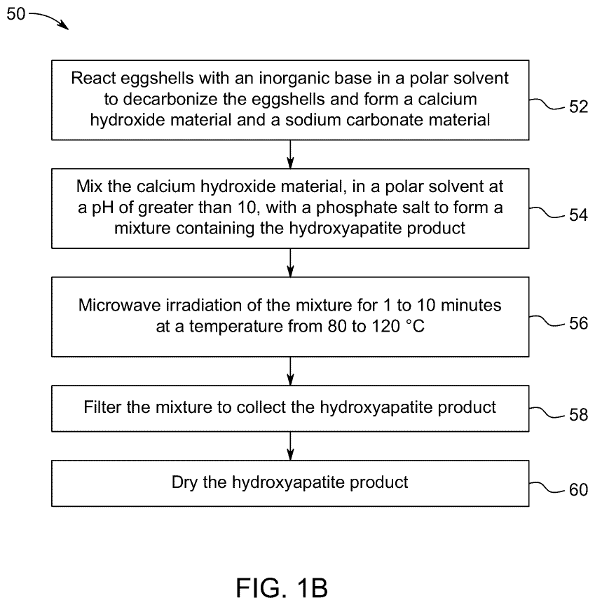

FIG. 1B illustrates a flow chart of a method 50 for synthesizing a hydroxyapatite (HAp) product. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes reacting eggshells with an inorganic base in a polar solvent to decarbonize the eggshells and form a calcium hydroxide material and a sodium carbonate material. Eggshells mainly constitute calcite, calcium carbonate (˜94 wt. %), magnesium carbonate (˜1 wt. %), calcium phosphate (˜1 wt. %), and organic debris (˜4 wt. %). The eggshells are in the form of a powder with an average crystallite size of 60-90 nm, preferably 65-85 nm, preferably 70-80 nm, and more preferably 72-76 nm. In a preferred embodiment, the average crystallite size of eggshell powder is about 74 nm.

The eggshells react with an inorganic base. Suitable examples of inorganic bases include, but are not limited to, beryllium hydroxide (Be(OH)₂), magnesium hydroxide (Mg(OH)₂), strontium hydroxide (Sr(OH)₂), calcium hydroxide (Ca(OH)₂), lithium hydroxide (LiOH), sodium hydroxide (NaOH), potassium hydroxide (KOH), rubidium hydroxide (RbOH), cesium hydroxide (CsOH), sodium bicarbonate (NaHCO₃), potassium bicarbonate (KHCO₃), and the like. In an embodiment, the inorganic base is a hydroxide base. In a preferred embodiment, the hydroxide base is NaOH. In some embodiments, the inorganic base may be a combination of one or more inorganic bases.

Polar solvents are solvents containing partial positive charge and partial negative charge. Suitable examples of polar solvents include, but are not limited to, water, methanol, ethanol, acetone, dimethyl sulfoxide (DMSO), dimethylformamide, dimethylacetamide, isopropanol, and the like. The water may be tap water, distilled water, bidistilled water, deionized water, deionized distilled water, reverse osmosis water, and/or some other water. In a preferred embodiment, the polar solvent is water. In a preferred embodiment, the water is distilled water. In some embodiments, the reaction occurs at an ambient temperature and an ambient pressure for 5-20 minutes (min), preferably 6-19 min, preferably 7-18 min, preferably 8-17 min, preferably 9-16 min, preferably 10-15 min, preferably 11-14 min, and preferably 12-13 min. In some embodiments, the ambient temperature is in the range of 20 to 30° C., preferably 21 to 29° C., preferably 22 to 28° C., preferably 23 to 27° C., preferably 24 to 26° C., and more preferably about 25° C. In some embodiments, the ambient pressure is in the range of 0.8 to 1.2 atmospheres (atm), preferably 0.9 to 1.1 atm, and more preferably about 1.0 atm.

The decarbonization reaction takes place, resulting in the formation of calcium hydroxide (Ca(OH)₂) and sodium carbonate (Na₂CO₃). In some embodiments, 94-98 percent by weight (wt. %), preferably 94.5-97.5 wt. %, preferably 95-97 wt. %, preferably 95.5-96.5 wt. %, and preferably about 96 wt. %, of the eggshells is converted into the Ca(OH)₂ material and the Na₂CO₃ material based on an initial weight of the eggshells. In a preferred embodiment, 96 wt. % of eggshells get converted into Ca(OH)₂ and Na₂CO₃. In some embodiments, the Ca(OH)₂ material and Na₂CO₃ material are separated by filtration. In some embodiments, the filtration may include vacuum filtration, cold filtration, centrifugal filtration, gravity filtration, granular media filtration, mechanical filtration, the like, an a combination thereof. In some embodiments, the decarbonization reaction may be dried, including removing any excess polar solvents and removing any excess inorganic base. In some embodiments, removing any excess inorganic base may be done with a polar solvent. In a preferred embodiment, the polar solvent is methanol. In some embodiments, the Na₂CO₃ material may be filtered and remain in a filtrate and may be dried. The Ca(OH)₂ material may be in a retentate and may be dried. The obtained dried calcium hydroxide powder was used as the calcium source precursor for the HAp product. In some embodiments, a weight ratio of the eggshells to the inorganic base to the polar solvent is from 5:30:50 to 10:40:60, preferably 5.5:31:51 to 9.5:39:59, preferably 6:32:52 to 9:38:58, preferably 6.5:33:53 to 8.5:37:57, and preferably 6:34:54 to 8:36:56 based on a percent by weight of a total weight of the reacting. In a preferred embodiment, the weight ratio of the eggshells to the inorganic base to the polar solvent is about 8.1:37.3:54.6. In some embodiments, a weight ratio of the eggshells to the inorganic base is from 1:4 to 1:6, preferably 1:4.3 to 1:5.7, preferably 1:4.5 to 1:5.5, and preferably 1:4.7 to 1:5.3, based on a weight of the eggshells and the inorganic base in the reacting mixture. In a preferred embodiment, the weight ratio of the eggshells to the inorganic base is about 1:4.61 based on a weight of the eggshells and the inorganic base in the reacting mixture. In some embodiments, a weight ratio of the inorganic base to the polar solvent is from 3:5 to 6:7, preferably 3.1:5.1 to 5.9:6.9, preferably 3.2:5.2 to 5.8:6.8, preferably 3.3:5.3 to 5.7:6.7, preferably 3.4:5.4 to 4.6:6.6, and preferably 3.5:5.5 to 4.5:5.5 based on a weight of the inorganic base and the polar solvent in the reacting mixture. In a preferred embodiment, the weight ratio of the inorganic base to the polar solvent is 4.61:6.75 based on a weight of the inorganic base and the polar solvent in the reacting mixture. In some embodiments, a weight ratio of the eggshells to the polar solvent is 1:6 to 1:10, preferably 1:6.5 to 1:9.5, preferably 1:7 to 1:9, and preferably 1:7.5 to 1:8.5 based on a weight of the eggshells and the polar solvent in the reacting mixture. In a preferred embodiment, the ratio of the eggshells to the polar solvent is about 1:6.75 based on a percent by weight of the total weight of the reacting mixture.

At step 54, the method 50 includes mixing the calcium hydroxide material, in a polar solvent at a pH of greater than 10, more preferably at a pH of about 11, with a phosphate salt to form a mixture including the HAp product. Suitable examples of phosphate salt include anhydrous monosodium phosphate, monosodium phosphate monohydrate or monosodium phosphate dihydrate, anhydrous disodium phosphate, disodium phosphate dihydrate, phosphorus disodium acid heptahydrate, disodium phosphate octahydrate or disodium phosphate decahydrate, hexagonal anhydrous trisodium phosphate, cubic anhydrous trisodium phosphate, trisodium phosphate hemihydrate hydrate, trisodium phosphate hexahydrate, trisodium phosphate octahydrate, trisodium phosphate dodecahydrate, ammonium dihydrogen phosphate, and the like. In a preferred embodiment, the phosphate salt is ammonium dihydrogen phosphate (NH₄H₂PO₄). In some embodiments, the phosphate salt may include a combination of one or more phosphate salts. In some embodiments, a ratio of the calcium hydroxide material to the phosphate salt is from 1:1 to 5:1, preferably 1.5:1 to 4.5:1, preferably 2:1 to 4:1, preferably 2.5:1 to 3.5:1, and more preferably about 3:1. In a preferred embodiment, the ratio of the calcium hydroxide material to the phosphate salt is 3:1. In a preferred embodiment, a molar ratio of calcium to phosphorus is about 1.67:1

At step 56, the method 50 includes microwave irradiating the mixture for 1-10 minutes, preferably 2-9 minutes, preferably 3-8 minutes, preferably 4-7 minutes, preferably 5-6 minutes, and more preferably about 5 minutes at a temperature from 80-120° C., preferably 85-115° C., preferably 90-110° C., preferably 95-105° C., and more preferably about 95° C. In some embodiments, microwave irradiating occurs at a frequency of 2-3 GHZ, preferably 2.1-2.9 GHZ, preferably 2.2-2.8 GHz, preferably 2.3-2.7 GHz, preferably 2.4-2.6 GHZ, preferably 2.45-2.55 GHz, and more preferably about 2.55 GHz. In some embodiments, the microwave irradiating occurs at a power of 600-1000 watts (W) preferably 610-990 W, preferably 620-980 W, preferably 630-970 W, preferably 640-960 W, preferably 650-950 W, preferably 660-940 W, preferably 670-930 W, preferably 680-920 W, preferably 690-910 W, preferably 700-900 W, preferably 710-890 W, preferably 720-880 W, preferably 730-870 W, preferably 740-860 W, preferably 750-850 W, preferably 760-840 W, preferably 770-830 W, preferably 780-820 W, preferably 790-810 W, and more preferably about 800 W. In a preferred embodiment, the mixture is exposed to microwave irradiation at 2.45 GHz using 800 W for 5 minutes at a temperature of 95° C.

At step 58, the method 50 includes filtering the mixture to collect the HAp product. Suitable techniques for separation include centrifugation, internal and external filtration, natural and forced sedimentation, magnetic separation, vacuum filtration, vacuum distillation, chemical conversion, the like, and a combination thereof. In a preferred embodiment, the separation of the mixture is done by vacuum filtration.

At step 60, the method 50 includes drying the HAp product. The HAp product is dried using heating appliances such as ovens, microwaves, autoclaves, hot plates, heating mantles and tapes, oil baths, salt baths, sand baths, air baths, hot-tube furnaces, hot-air guns, the like, and combinations thereof. In some embodiments, the HAp product is dried at 100-120° C., more preferably 105-115° C., for 1-5 hours (h), preferably 2-4 hours, and more preferably 2.5-3.5 hours. In a preferred embodiment, the HAp product is dried in a conventional oven at about 110° C. for about 3 hours.

In some embodiments, the HAp product has a calcium-to-phosphorous molar ratio of 1.5-1.9, preferably 1.55-1.85, preferably 1.6-1.8, preferably 1.65-1.75, and most preferably about 1.67. In some embodiments, the HAp product contains carbon in an amount of 15-25 at. %, preferably 16-24 at. %, preferably 17-23 at. %, preferably 18-22 at. %, and more preferably 19-21 at. %; oxygen in an amount of 60-70 at. %, preferably 61-69 at. %, preferably 62-68 at. %, preferably 63-67 at. %, and more preferably 64-66 at. %; calcium in an amount of 15-25 at. %, preferably 16-24 at. %, preferably 17-23 at. %, preferably 18-22 at. %, and more preferably 19-21 at. %; and phosphorous in an amount of 3-10 at. %, preferably 4-9 at. %, preferably 5-8 at. %, and more preferably 6-7 at. %, based on a total atom count of the hydroxyapatite product. In a preferred embodiment, the HAp product contains carbon in an amount of about 18.4 at. %, oxygen in an amount of about 62.89 at. %, calcium in an amount of about 11.7 at. %, and phosphorous in an amount of about 7.01 at. %, based on a total atom count of the hydroxyapatite product.

In some embodiments, the HAp product particles may exist in various morphological shapes, such as rods, spheres, wires, crystals, rectangles, triangles, pentagons, hexagons, prisms, disks, cubes, ribbons, blocks, beads, toroids, discs, barrels, granules, whiskers, flakes, foils, powders, boxes, stars, tetrapods, belts, flowers, the like, and mixtures thereof. In a preferred embodiment, the HAp product has a particle shape in the form of nanorods. In some embodiments, the nanorods are agglomerated.

In some embodiments, the nanorods have an average size of 10-40 nm, preferably 11-39 nm, preferably 12-38 nm, preferably 13-37 nm, preferably 14-36 nm, preferably 15-35 nm, preferably 16-34 nm, preferably 17-33 nm, preferably 18-32 nm, preferably 19-31 nm, preferably 20-30 nm, preferably 21-29 nm, preferably 22-28 nm, preferably 23-27 nm, preferably 24-26 nm, and more preferably about 25 nm. In some embodiments, the nanorods have the longest dimension of 10-150 nm, preferably 20-140 nm, preferably 30-130 nm, preferably 40-120 nm, preferably 50-110 nm, preferably 60-100 nm, and more preferably 70-90 nm.

In some embodiments, the HAp product has a crystalline apatite structure. In X-ray diffraction (XRD), the interplanar spacing (d-spacing) of a crystal is used for identification and as characterization objectives. The d-spacing is the distance between atomic planes that produce diffraction peaks. Each peak in a diffractogram results from a corresponding d-spacing. The planes of atoms can be referred to as a three-dimensional coordinate system and can be described as a direction within the crystal. In some embodiments, the HAp product has a first d-spacing value of 0.330-0.340 nm, preferably 0.331-0.339 nm, preferably 0.332-0.338 nm, preferably 0.333-0.337 nm, preferably 0.334-0.336 nm, and more preferably about 0.335 nm. In some embodiments, the HAp product has a second d-spacing value of 0.280-0.285 nm, preferably 0.281-0.284 nm, preferably 0.282-0.283 nm, and more preferably about 0.281 nm.

### EXAMPLES

The following examples demonstrate a method for synthesizing a hydroxyapatite (HAp) product. The examples are provided solely for illustration and are not to be construed as limitations of the present disclosure, as many variations thereof are possible without departing from the spirit and scope of the present disclosure.

### Example 1: Materials and Methods

Eggshell waste material was collected from the King Fahd University of Petroleum and Minerals (KFUPM) food court restaurant, where it was later subjected to tap water washing several times before it was dried at 110° C. for 3 hours (h) in an oven. The dried waste was crushed in an electric motor blender for 30 minutes (min) before it was sieved and passed through a 100-mesh sieve (˜149μ) to form a homogenous powder. The prepared powder was then used for the decarbonization step, where specific amounts of eggshell powder were mixed with sodium hydroxide pellets (from Sigma Aldrich) and distilled water, as shown in Table 2, to achieve a maximum conversion percentage of ˜96% of eggshell waste into calcium hydroxide (Ca(OH)₂) and sodium carbonate (Na₂CO₃), as the resulting materials from this decarbonization process, as per the following chemical reaction:

(Eggshell) CaCO₃(s)+2NaOH(aq)+xH₂O→Ca(OH)₂+Na₂CO₃·xH₂O (x=0 or 1)

The previous parameters shown in Table 2 were adapted from the study by Hanein and coworkers, [Hanein, T. et al., Decarbonization of calcium carbonate at atmospheric temperatures and pressures, with simultaneous CO₂ capture, through the production of sodium carbonate. Energy & Environmental Science, 2021. 14(12): p. 6595-6604, which is incorporated herein by reference in its entirety] where the decarbonization of commercial CaCO₃ at atmospheric temperatures and pressures was investigated and described in more detail in which different starting proportions of CaCO₃, NaOH, and H₂O were studied to achieve different sufficient conversion percentages.

The decarbonization was done at ambient conditions, using Table 2 parameters, with continuous mixing using a magnetic stirrer in a 100 mL Teflon beaker for ˜10 minutes (min). A vacuum filtration system was used to separate the products via different steps. The unreacted excess NaOH was recovered, using methanol alcohol (methanol), from the other compounds.

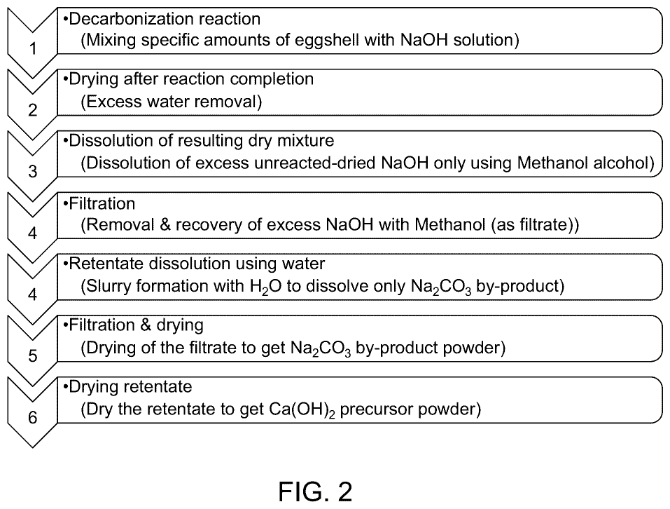

Later, with water preferential dissolution, the other two resulting precipitates (Ca(OH)₂ & Na₂CO₃) were obtained and dried separately, as shown in FIG. 2. The obtained dried sodium carbonate, soda ash, were then stored in a closed container for future usage. At the same time, the obtained dried calcium hydroxide powder was used as the calcium source precursor for the HAp synthesis using microwave processing.

TABLE 2
Used eggshell decarbonization parameters.

| Precursors | Eggshells | NaOH | H₂O | Conversion (%) to Ca(OH)₂ |
|---|---|---|---|---|
| Amount in (g) | 1.00 | 4.61 | 6.75 | 96% |
| wt. % | 8.1 | 37.3 | 54.6 | |

To prepare a mixture, 300 mL of Ca(OH)₂ solution extracted from the decarbonization process was slowly mixed drop wise with 100 mL of ammonium dihydrogen phosphate (NH₄H₂PO₄) reagent (purchased from Sigma Aldrich), as the phosphate source, while continuously stirring. The mixture's pH was maintained at around 11 using an Orion Star PH meter. The concentration of Ca²⁺ and PO₄³⁻ in the mixture was 0.7775 M and 0.4656 M, respectively, which correspond to the Ca/P molar ratio of 1.67, the theoretical molar ratio of HAp, according to the following reaction:

10Ca(OH)₂+6NH₄H₂PO₄→Ca₁₀(PO₄)₆(OH)₂+6NH₄OH+12H₂O

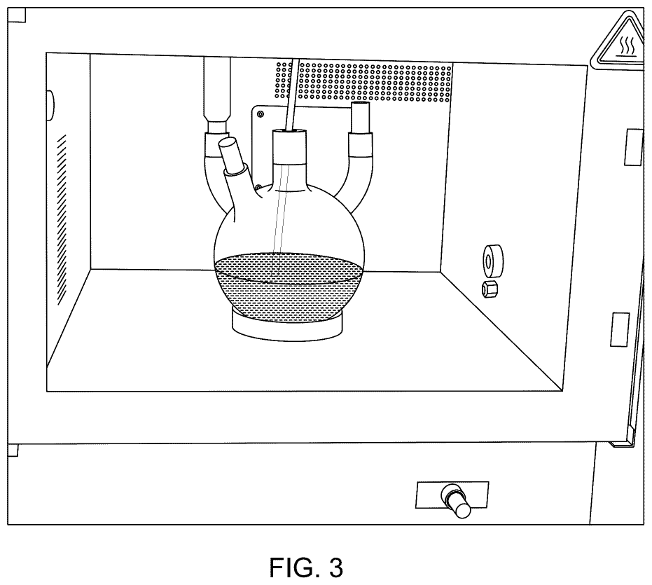

The mixture was transferred into a 4-neck flask and then directly inside a MW synthesis unit, Sineo MAS-II Plus, where the reaction mixture was exposed to MW radiation at 2.45 GHz for 5 minutes using 800 watts (W), where the temperature was controlled at around 95° C., with a continuous stirring during MW exposure time, as shown in FIG. 3.

After cooling, the sample was collected, filtered through the vacuum filtration system, and washed twice with distilled water before being dried in a conventional oven at 110° C. for 3 hours. The dried HAp resulting powder was then characterized using X-ray diffraction (XRD) with Rigaku Miniflex-II Mini-X-ray Diffraction machine (manufactured by Rigaku, Japan) at 2° min⁻¹ rate using Cu K_{α} source with 0.15406 nm wavelength where a 10 mA current and 30 kV voltage were used. Fourier-transform infrared (FTIR) analysis was performed on the Smart iTR NICOLET iS10 FTIR machine (manufactured by Thermo Fischer Scientific, United States), and wavenumber 4000 cm⁻¹ to 400 cm⁻¹ was scanned. Furthermore, the HAp powder was also characterized using a field emission scanning electron microscopy (FE-SEM) (model FEI Quanta 250) equipped with an energy-dispersive X-ray spectroscopy (EDS) detector (150 mm² Oxford detector) where the investigated powders were platinum coated using a Quorum coating machine model Q150R. A JEM-2100F field emission electron microscope (FE-TEM) with 200 kV was used to investigate the obtained HAp powder, where the powder was dispersed in 2-propanol/water and sonicated before testing.

### Results and Discussion

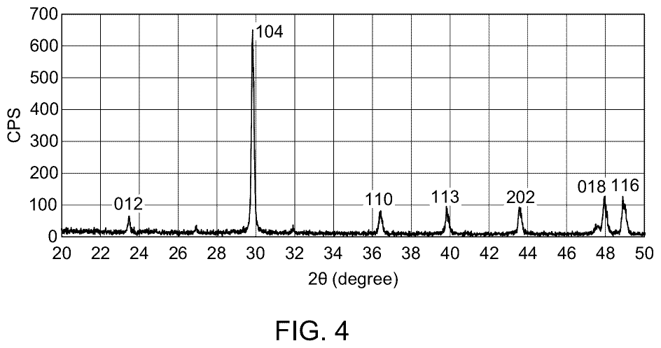

FIG. 4 shows the XRD pattern of eggshell powder after being cleaned, crushed, and sieved. The XRD peaks were indexed and identified as CaCO₃, as predicted, where the peaks match with the Joint Committee on Powder Diffraction Standards (JCPDS) file No. 05-0586 [Kumar, G. S., A. Thamizhavel, and E. K. Girija, Microwave conversion of eggshells into flower-like hydroxyapatite nanostructure for biomedical applications. Materials Letters, 2012. 76: p. 198-200, which is incorporated herein by reference in its entirety]. The average crystallite size of eggshell powder was calculated to be around 74 nm using the Scherrer equation as follows:

$$β = \frac{k λ}{L cos θ}$$

where β is the full-width at half-maximum (FWHM) of the diffraction peak in radians, L is crystalline size in (Å), k is 0.9, θ is the Bragg angle, and λ has a value of 1.5406 Å.

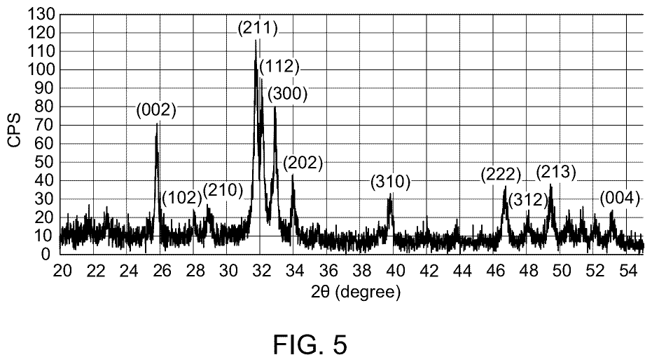

XRD pattern of microwave synthesized HAp powder using decarbonized eggshell is shown in FIG. 5, where the 2θ scan range was between 20° and 55°. The existence of the unique, intense peak at 2θ=31.75° for the (211) plane is identified as a characteristic peak for the apatite formation, in addition to the other remaining peaks related to HAp structure. the XRD pattern, therefore, indicates the successful formation of a single pure crystalline hexagonal HAp phase, where it is indexed and identified by JCPDS (09-0432) and matches well with the literature [Khalid, M., et al., Synthesis and characterizations of hydroxyapatite using precursor extracted from chicken eggshell waste. Biointerface Research in Applied Chemistry, 2022. 12(4): p. 5663-5671; Sari, Y. W., et al., Effects of microwave processing parameters on the properties of nanohydroxyapatite: Structural, spectroscopic, hardness, and toxicity studies. Ceramics International, 2021. 47(21): p. 30061-30070; and Kalbarczyk, M. and A. Szcześ, Potential biomedical application of calcium phosphates obtained using eggshells as a biosource of calcium at different initial pH values. Ceramics International, 2021. 47(23): p. 33687-33696; each or which are incorporated herein by references in their entireties]. The average crystal size for synthesized HAp was calculated to be around 28 nm using the Sheerer equation. Microwave synthesis of the decarbonized eggshells successfully produced pure nano-sized HAp, as revealed by the XRD pattern.

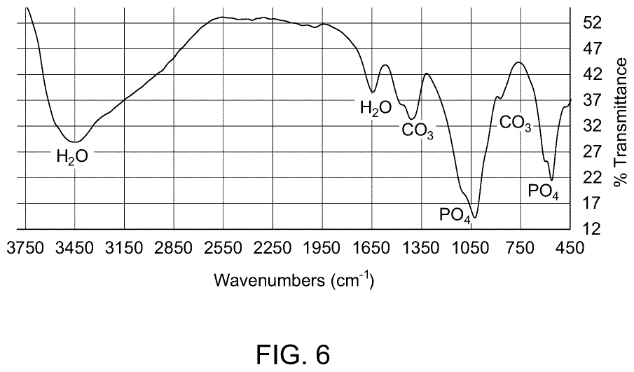

The FTIR spectra of the synthesized HAp are shown in FIG. 6. The absorption bands at around 1430 cm⁻¹ and 867 cm⁻¹ are assigned to the presence of carbonate ions in the HAp sample, which is due to the reaction of atmospheric carbon dioxide with HAp during synthesis. The unique [PO₄]³⁻ stretching mode is observed at 1022 cm⁻¹, while the other bending modes of [PO₄]³⁻ are observed at around 565 cm⁻¹ and 585 cm⁻¹. The peaks around 1640 cm⁻¹ and 3430 cm⁻¹ are due to the vibration modes of the H₂O molecule, which matches what was reported in the literature [Ganesan, V., et al., Eggshell derived mesoporous biphasic calcium phosphate for biomedical applications using rapid thermal processing. International Journal of Applied Ceramic Technology, 2019. 16(5): p. 1932-1943; and Ashokan, A., et al., Eggshell derived hydroxyapatite microspheres for chromatographic applications by a novel dissolution—precipitation method. Ceramics International, 2021. 47(13): p. 18575-18583, both of which are incorporated herein by references in their entirety].

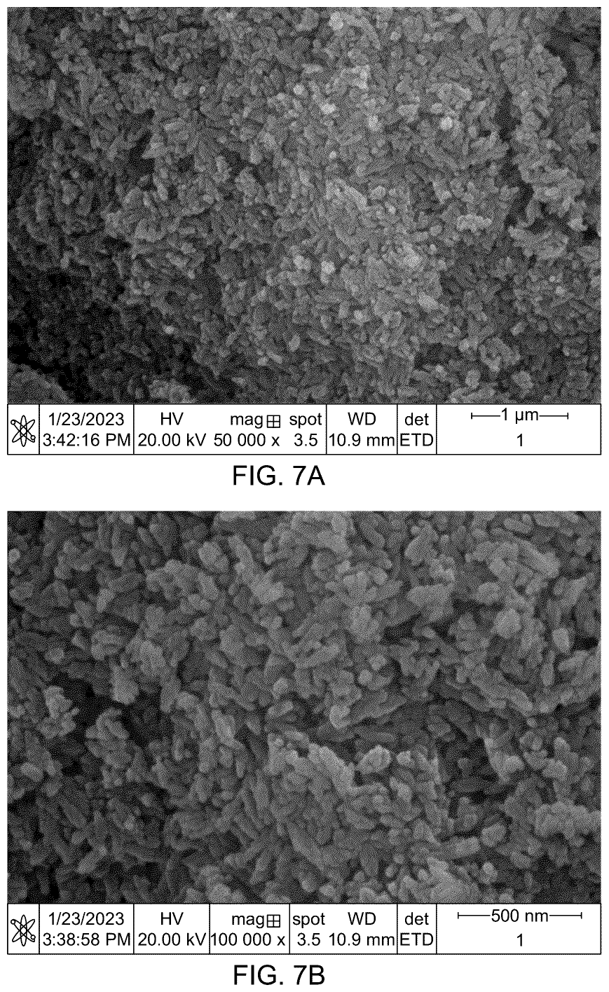

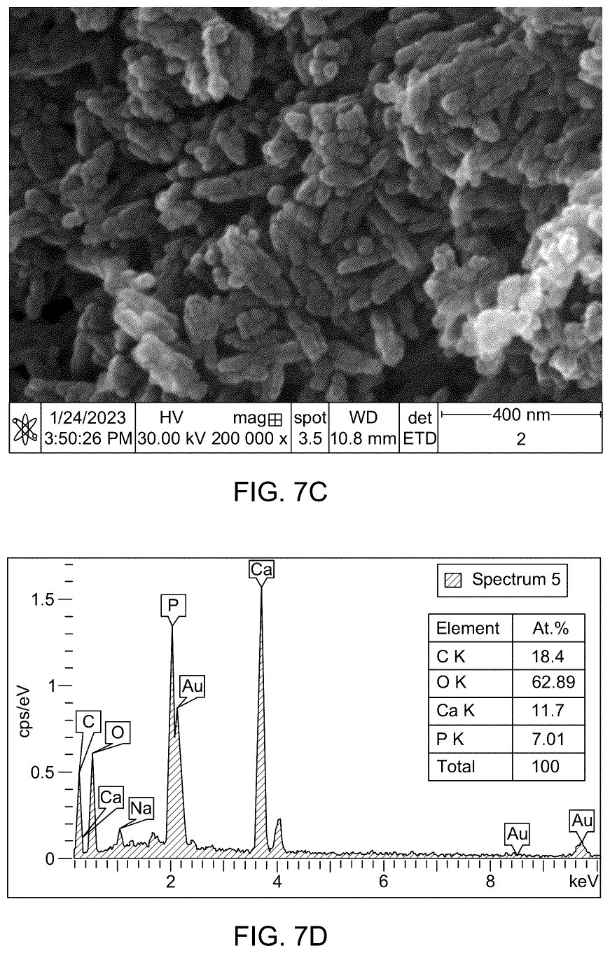

SEM micrographs of the MW synthesized HAp from decarbonized eggshells are shown in FIGS. 7A-7C. The morphology of the synthesized HAp appears to be an aggregation of individual particles with platelet-like (rods and/or needle) shape in the nano-size range as predicated from the sheerer equation using the XRD pattern. The individual particles' exact shape could not be easily recognized from SEM micrographs due to their small nano-size. EDX analysis is shown in FIG. 7D, where the peaks of calcium, phosphorous, and oxygen indicate the synthesis of HAp with the exact Ca/P ratio of 1.67, as designed in the synthesis step.

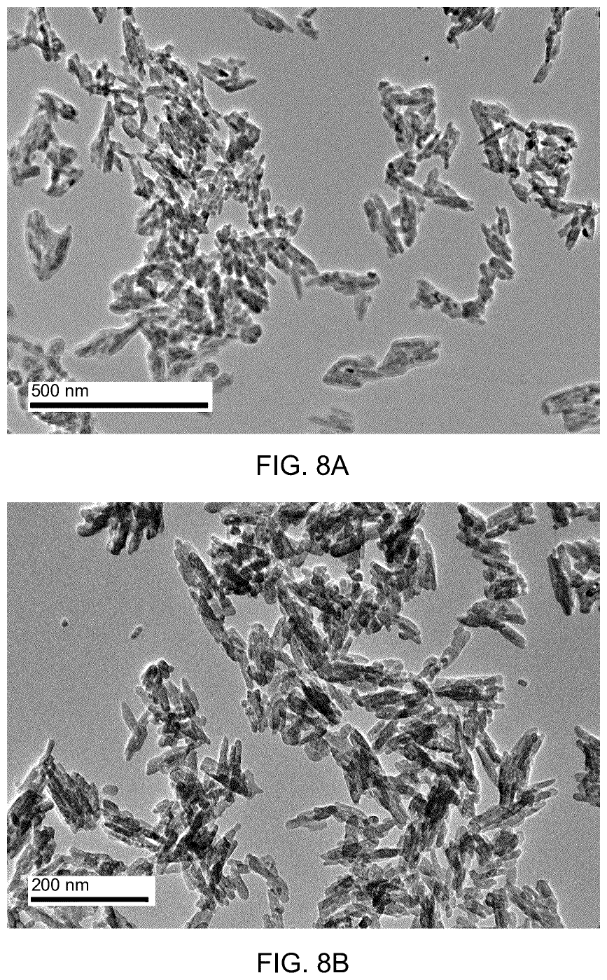

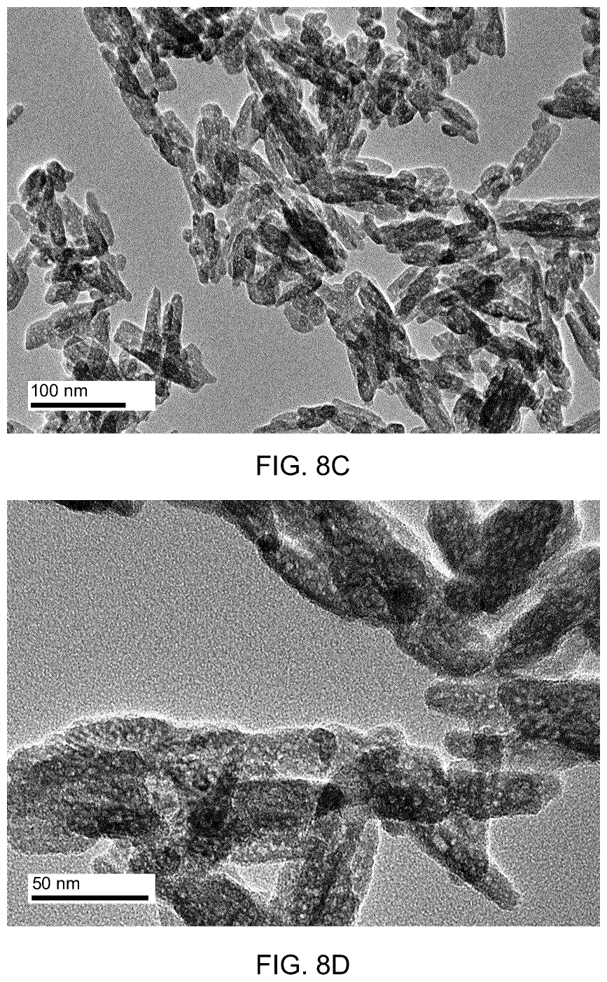

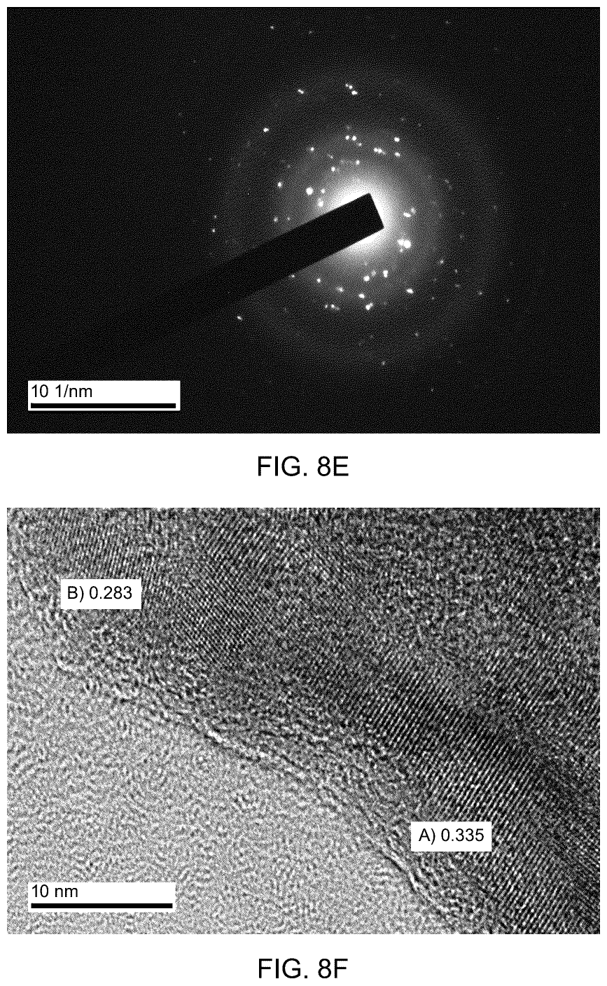

TEM images of the MW synthesized HAp are shown in FIGS. 8A-8F. The images reveal a highly crystalline apatite structure without the presence of any other crystalline phases, which is consistent with the XRD and FTIR analyses. TEM images clearly show the HAp particle shape as highly agglomerated and elongated nano-rods with an average size of around 25±5 nm. The selected area electron diffraction (SAED) pattern (FIG. 8E) exhibits concentric rings that represent the presence of the polycrystalline structure of MW-synthesized HAp, which agrees with the XRD pattern. The interplanar lattice fringe spacing of the MW synthesized HAp using decarbonized eggshells was measured as in FIG. 8F. At point A, the d-spacing is calculated to be 0.335 nm, which corresponds to the (0002) plane, the middle basal plane of the HAp hexagonal unit cell. This is in closer agreement with an earlier study investigating an apatite hexagonal cell structure where a d-spacing value of 0.34 nm for the same plane was reported [Cuisinier, F., et al., Transmission electron microscopy of lattice planes in human alveolar bone apatite crystals. Calcified Tissue International, 1987. 40(6): p. 332-338, which is incorporated herein by reference in its entirety]. Furthermore, the d-spacings for the (002) plane of the crystalline particles were reported to be 0.34 nm in another investigation for a needle-shaped apatite crystal structure [Hong, S. I., et al., Ultrastructural analyses of nanoscale apatite biomimetically grown on organic template. J Mater Res., 2008. 23(2): p. 478-485, which is incorporated herein by reference in its entirety]. Point B in FIG. 8F shows a measured value of 0.283 nm that closely corresponds to (2111) lattice plane, which is also in close agreement with the same earlier study [Cuisinier, F., et al., Transmission electron microscopy of lattice planes in human alveolar bone apatite crystals. Calcified Tissue International, 1987. 40(6): p. 332-338, which is incorporated herein by reference in its entirety] where they reported a d-spacing of 0.281 nm for that same lattice plane of a hexagonal apatite cell. Furthermore, the d-spacing between lattice fringes of needle-shaped apatite crystal plate particles at (211) or (112) planes, the two major XRD peaks for HAp, were reported to be in the range of 0.27-0.29 nm [Hong, S. I., et al., Ultrastructural analyses of nanoscale apatite biomimetically grown on organic template. J Mater Res., 2008. 23(2): p. 478-485, which is incorporated herein by reference in its entirety], which matches the measured point B value in FIG. 8F of the MW synthesized nano-sized HAp.

The decarbonization process of eggshell waste was achieved successfully with a high conversion percentage using an alkaline sodium hydroxide solution treatment, resulting in two valuable by-products. The first was calcium hydroxide, which was used as a calcium source precursor for nano-HAp preparation using microwave (MW) synthesis, and the second was soda ash (Na₂CO₃). Decarbonization will not only transfer eggshell waste into valuable biomaterial, such as nano-HAp, but also store and trap carbon dioxide as a global warming and climate change mitigation factor into a valuable by-product, soda ash (Na₂CO₃). Soda ash is used in vast industrial activities, such as ceramics, glasses, detergents, and chemicals, and global reserves are minimal and not geographically widespread. The process of eggshell decarbonization is economically and environmentally appealing since almost 1 gram of CaCO₃ is going to produce a nearly equal amount of such soda ash and Ca(OH)₂ precursor that is going to be converted later into nano-sized HAp material as a promising biomaterial. The decarbonization process of eggshells will contribute to the concept of a circular economy and sustainability demands for better human life. MW synthesis was used to successfully prepare nano-HAp via the reaction of decarbonized eggshell by-product, (Ca(OH)₂, with NH₄H₂PO₄. A single phase of pure nano-sized HAp biomaterial with an average crystal size of 28 nm was prepared in a short time (5 minutes). The prepared nano-HAp was characterized using XRD, FTIR, and electron microscopy (SEM-EDX and TEM), where an agglomeration of individual particles with a rod-like shape was observed and identified with a polycrystalline hexagonal crystal structure of HAp. EDX data also confirmed the formation of HAp with its unique Ca/P ratio of 1.67. The d-spacings of the major lattice planes in the hexagonal HAp lattice cell and its particles were measured and identified using FE-TEM.

Numerous modifications and variations of the present disclosure are possible in light of the above teachings. It is therefore to be understood that within the scope of the appended claims, the disclosure may be practiced otherwise than as specifically described herein.

1: A method of synthesizing a hydroxyapatite product, including:
reacting eggshells with an inorganic base in a polar solvent to decarbonize the eggshells and form a calcium hydroxide material and a sodium carbonate material,
mixing the calcium hydroxide material, in a polar solvent at a pH of greater than 10, with a phosphate salt to form a mixture including the hydroxyapatite product,
wherein a ratio of the calcium hydroxide material to the phosphate salt is from 1:1 to 5:1,
microwave irradiating the mixture for 1 to 10 minutes (min) at a temperature from 80 to 120 degree Celsius (° C.);
filtering the mixture to collect the hydroxyapatite product; and
drying the hydroxyapatite product.

2: The method of claim 1, wherein 94 to 98 percent by weight of the eggshells is converted into the calcium hydroxide material and the sodium carbonate material based on an initial weight of the eggshells.

3: The method of claim 1, wherein the hydroxyapatite product has a particle shape in the form of nanorods.

4: The method of claim 3, wherein the nanorods are agglomerated.

5: The method of claim 3, wherein the nanorods have an average size of 10 to 40 nanometers (nm).

6: The method of claim 3, wherein the nanorods have a longest dimension of 10 to 150 nm.

7: The method of claim 1, wherein the hydroxyapatite product includes carbon in an amount of 15 to 25 atomic percent (at. %), oxygen in an amount of 60 to 70 at. %, calcium in an amount of 5 to 15 at. %, and phosphorous in an amount to 3 to 10 at. % based on a total atom count of the hydroxyapatite product.

8: The method of claim 1, wherein the hydroxyapatite product has a crystalline apatite structure.

9: The method of claim 1, wherein the hydroxyapatite product has a calcium-to-phosphorous molar ratio of 1.5 to 1.9.

10: The method of claim 1, wherein the hydroxyapatite product has a first d-spacing value of 0.330 to 0.340 nm.

11: The method of claim 1, wherein the hydroxyapatite product has a second d-spacing value of 0.280 to 0.285 nm.

12: The method of claim 1, wherein the eggshells are in the form of a powder with an average crystallite size of 60 to 90 nm.

13: The method of claim 1, wherein the reacting occurs at ambient temperature and ambient pressure for 5 to 20 minutes.

14: The method of claim 1, wherein a ratio of the eggshells to the inorganic base to the polar solvent is from 5:30:50 to 10:40:60 based on a percent by weight of a total weight of the reacting.

15: The method of claim 1, wherein the calcium hydroxide material and the sodium carbonate material are separated by filtration.

16: The method of claim 1, wherein the microwave irradiating occurs at a frequency of 2 to 3 gigahertz (GHz).

17: The method of claim 1, wherein the microwave irradiating occurs at a power of 600 to 1000 watts (W).

18: The method of claim 1, wherein the phosphate salt is ammonium dihydrogen phosphate.

19: The method of claim 1, wherein the inorganic base is a hydroxide base.

20: The method of claim 1, wherein the drying occurs at 100 to 120° C. for 1 to 5 hours (h).
